(19) United States
(12) Patent Application Publication
(10) Pub. No.: US 2008/0194695 A1
(43) Pub. Date: Aug. 14, 2008

(54) Pharmaceutical Composition Containing Sibutramine Free Base and Manufacturing Method Thereof

(76) Inventors: Hong-Ryeol Jeon, Gyeonggi-do (KR); Do-Woo Kwon, Chungcheongnam-do (KR); Bong-Sang Lee, Gyeonggi-do (KR); Se-Heum Oh, Gyeonggi-do (KR); Dong-Ryun Oh, Gyeonggi-do (KR); Jeong-Seo Park, Gyeonggi-do (KR); Se-Geun Yu, Gyeonggi-do (KR)

Correspondence Address: STROOCK & STROOCK & LAVAN LLP, 180 MAIDEN LANE, NEW YORK, NY, 10038, US

(21) Appl. No.: 11/912,081

(22) PCT Filed: Apr. 18, 2006
(86) PCT No.: PCT/KR2006/001434

§ 371 Date: Oct. 19, 2007

(30) Foreign Application Priority Data
Apr. 20, 2005 (KR) 10-2005-0032744
Feb. 2, 2006 (KR) 10-2006-0010046

(51) Int. Cl. A61K 31/135; A61P 25/00

(52) U.S. Cl. 514/646

(57) ABSTRACT

The present invention relates to a composition comprising sibutramine free base. The present invention provides a solid dispersion wherein sibutramine free base, acid and hydrophilic polymer are uniformly dispersed and a manufacturing method thereof. The composition of the present invention has improved dissolution rate compared to conventional compositions containing sibutramine free base or sibutramine hydrochloride, and is also stable during storage and can be easily mass-produced.

### TECHNICAL FIELD

The present invention relates to a composition comprising sibutramine free base and manufacturing method thereof.

### BACKGROUND ART

Sibutramine is a common name of N,N-dimethyl-1-[1-(4-chlorophenyl)-cyclobutyl]-3-methylbutylamine, and known as being used for treating or preventing depression, Parkinson disease and obesity (See British Patent No. 2,098,602 and PCT international publication No. WO 88/06444). Further, sibutramine may be used for reducing the resistance to insulin or enhancing the resistance to sugar, and for preventing or treating such diseases as gout, hyperuricemia, hyperlipemia, osteoarthritis, anxiety disorder, somnipathy, sexual dysfunction, chronic fatigue syndrome and cholelithiasis (see U.S. Pat. Nos. 6,174,925, 5,459,164, 6,187,820, 6,162,831, 6,232,347, 6,355,685, 6,365,631, 6,376,554, 6,376,551 and 6,376,552).

The solubility of sibutramine free base rapidly decreases according to the increase of pH of dissolution medium so that sibutramine free base does not almost dissolve in distilled water and pH 6.8 buffered solution, which means that there may be a problem in body absorption of sibutramine free base, that is, bioavailability. Zohreh Abolfathi et al. reported that intake of food severely changes the area under the curve of blood concentration (AUC), the maximum blood concentration (Cmax) and the time required for the maximum blood concentration (Tmax) (“A pilot study to evaluate the pharmacokinetics of sibutramine in healthy subjects under fasting and fed conditions”, J Pharm Pharmaceut Sci, 7(3):345-349, 2004), and these changes of the pharmacokinetic data are expected to be caused by the changes of sibutramine hydrochloride solubility and gastric emptying time which made by food intake, even if there may be other reasons. The change of gastric emptying time can be also thought to be related with the solubility of sibutramine hydrochloride because the big pH difference between stomach and upper intestine causes the solubility difference of sibutramine hydrochloride, which causes the difference of absorption or bioavailability. The absorption variation of sibutramine free base may be bigger than sibutramine hydrochloride because the solubility difference of sibutramine free base is much bigger than sibutramine hydrochloride evaluated by Zohreh Abolfathi et al.

In addition, sibutramine free base may become sticky gel phase during storage, that is, the stability of sibutramine free base is so bad that it is difficult to make a sibutramine free base-containing preparation by conventional manufacturing methods.

To solve these problems, British Patent No. 2,098,302 and Korean Patent Publication No. 90-00274 disclose methods for preparing sibutramine hydrochloride anhydrous as a pharmaceutically acceptable acid-addition salt. However, the sibutramine hydrochloride anhydrous is highly hygroscopic, so that it is difficult to keep the content of the active ingredient constant, and absorbed water may cause hydrolysis or chemical degradation of the active ingredient. Accordingly, it is difficult to use the sibutramine hydrochloride anhydrous in a pharmaceutical composition (See U.S. Pat. No. 6,900,245).

To solve said problems, non-hygroscopic sibutramine hydrochloride sibutramine hydrochloride monohydrate was developed (See British Patent No. 2,184,122 and Korean Patent Publication No. 94-08913) and the sibutramine hydrochloride monohydrate is now used as an active ingredient in Meridia™ or Reductil™ for treating obesity.

Then again, U.S. Pat. No. 6,900,245 said that an active ingredient used in a pharmaceutical composition should be soluble in water or water solution having broad range of pH to guarantee in vivo dissolution rate (consequently, bioavailability) and from this point of view, the solubility of sibutramine hydrochloride monohydrate is insufficient for being used as an active ingredient, so that sibutramine methanesulfonate hemihydrate having improved solubility was developed. As shown in U.S. Pat. No. 6,900,245, the dissolution rate of a preparation containing sibutramine hydrochloride monohydrate in pH 6.8 buffered solution was evaluated to be not good.

However, all of the above methods are to make a salt through additional synthesis steps from sibutramine free base, which may increase manufacturing cost. In addition, the salt should be separated when a preparation containing sibutramine salt is evaluated. Further, it is preferable for reducing potential side-effects to decrease the content of additional ingredients including acid (for example, methanesulfonic acid) used in making a salt.

### DISCLOSURE OF INVENTION

### Technical Problem

Therefore, the object of the present invention is to provide a sibutramine free base-containing composition that has good dissolution property and little dissolution variation according to pH. In addition, the object of the present invention is to provide a sibutramine free base-containing composition that can be easily mass-produced and is stable. Another object of the present invention is to provide manufacturing method thereof.

### Technical Solution

To achieve the object, the present invention provides a sibutramine free base-containing solid dispersion wherein sibutramine free base, acid and hydrophilic polymer are uniformly dispersed.

More preferably, the present invention provides said sibutramine free base-containing solid dispersion wherein the acid is at least one selected from the group consisting of citric acid, fumaric acid, lactic acid, tartaric acid, succinic acid, maleic acid, malic acid, oxalic acid and phosphoric acid.

More preferably, the present invention provides said sibutramine free base-containing solid dispersion wherein the hydrophilic polymer is at least one selected from the group consisting of hydroxypropylcellulose, hydroxypropylmethylcellulose, polyvinylpyrrolidone, vinylpyrrolidone-vinylacetate copolymer, polyvinylalcohol and polyvinylalcohol-polyethyleneglycol copolymer.

More preferably, the present invention provides said sibutramine free base-containing solid dispersion wherein the acid is comprised in an amount of 0.1-20 moles per mole of the sibutramine free base.

More preferably, the present invention provides said sibutramine free base-containing solid dispersion wherein the acid is comprised in an amount of 0.06-10 parts by weight per part by weight of the sibutramine free base.

More preferably, the present invention provides said sibutramine free base-containing solid dispersion wherein the hydrophilic polymer is comprised in an amount of 0.125-30 parts by weight per part by weight of the sibutramine free base.

More preferably, the present invention provides said sibutramine free base-containing solid dispersion wherein the solid dispersion is coated by at least one selected from the group consisting of hydroxypropylmethylcellulose, vinylpyrrolidone-vinylacetate copolymer, polyvinylalcohol and polyvinylalcohol-polyethyleneglycol copolymer.

The present invention also provides a pharmaceutical preparation comprising any one of the sibutramine free base-containing solid dispersions.

The present invention also provides a method for manufacturing sibutramine free base-containing solid dispersion comprising (S1) making a solution wherein sibutramine free base, acid and hydrophilic polymer are dissolved; and (S2) drying the solution of (S1) step.

More preferably, the present invention provides said method for manufacturing sibutramine free base-containing solid dispersion wherein the acid is at least one selected from the group consisting of citric acid, fumaric acid, lactic acid, tartaric acid, succinic acid, maleic acid, malic acid, oxalic acid and phosphoric acid.

More preferably, the present invention provides said method for manufacturing sibutramine free base-containing solid dispersion wherein the hydrophilic polymer is at least one selected from the group consisting of hydroxypropylcellulose, hydroxypropylmethylcellulose, polyvinylpyrrolidone, vinylpyrrolidone-vinylacetate copolymer, polyvinylalcohol and polyvinylalcohol-polyethyleneglycol copolymer.

More preferably, the present invention provides said method for manufacturing sibutramine free base-containing solid dispersion wherein the acid is comprised in an amount of 0.1-20 moles per mole of the sibutramine free base.

More preferably, the present invention provides said method for manufacturing sibutramine free base-containing solid dispersion wherein the acid is comprised in an amount of 0.06-10 parts by weight per part by weight of the sibutramine free base.

More preferably, the present invention provides said method for manufacturing sibutramine free base-containing solid dispersion wherein the hydrophilic polymer is comprised in an amount of 0.125-30 parts by weight per part by weight of the sibutramine free base.

More preferable, the present invention provides said method for manufacturing sibutramine free base-containing solid dispersion wherein the method further comprises (S3) coating the solid dispersion of (S2) step with at least one selected from the group consisting of hydroxypropylmethylcellulose, vinylpyrrolidone-vinylacetate copolymer, polyvinylalcohol and polyvinylalcohol-polyethyleneglycol copolymer.

Hereinafter, sibutramine free base-containing composition and manufacturing method thereof according to the present invention will be described in detail.

The present invention provides a sibutramine free base-containing solid dispersion wherein sibutramine free base, acid and hydrophilic polymer are uniformly dispersed.

The solubility of sibutramine free base decreases rapidly according to increase of pH of dissolution medium, but the improved solubility and dissolution rate of the sibutramine free base contained in the solid dispersion of the present invention can keep even in high pH solution because acid and hydrophilic polymer in the solid dispersion are uniformly and minutely distributed around sibutramine free base to make micro-environmental condition acidic and keep the acidic micro-environments in dissolution medium. Further, the improving effect of the solid dispersion of the present invention is surprisingly great, so that it is possible to keep high dissolution rate even if acid and hydrophilic polymer are used in a small amount compared to the content of sibutramine free base.

In addition, the solid dispersion of the present invention improves the stability of severely hygroscopic sibutramine free base by making stable hydrophilic polymer surround sibutramine free base uniformly and minutely to block sibutramine free base from contacting exterior water.

The acid that may be used in the present invention includes, but is not limited to, organic acids such as citric acid, fumaric acid, lactic acid, tartaric acid, succinic acid, maleic acid, malic acid, oxalic acid, aspartic acid, glutamic acid, palmitic acid, propionic acid, ascorbic acid, chitic acid, hippuric acid, alginic acid, cholic acid, butyric acid, benzoic acid, methanesulfonic acid, benzenesulfonic acid, toluenesulfonic acid, salicylic acid, gluconic acid, glycolic acid, mandelic acid and cinnamic acid; inorganic acids such as hydrochloric acid, phosphoric acid, acetic acid, trifluoroacetic acid, hydrobromic acid and sulphuric acid; and their mixtures.

However, citric acid, fumaric acid, lactic acid, tartaric acid, succinic acid, maleic acid, malic acid, oxalic acid and phosphoric acid are preferable if considering manufacturing difficulty such as bad smell (for example, hydrochloric acid), solubility (for example, hippuric acid) and viscosity (for example, chitic acid and alginic acid), etc.; safety (for example, propionic acid, sulphuric acid and salicylic acid); and the improving degree of dissolution rate of solid dispersion.

The hydrophilic polymer that can be used in the present invention includes, but is not limited to, hydroxypropylcellulose, hydroxypropylmethylcellulose, polyvinylpyrrolidone, vinylpyrrolidone-vinylacetate copolymer, polyvinylalcohol, polyvinylalcohol-polyethyleneglycol copolymer, methylcellulose, ethylcellulose, hydroxymethylcellulose, hydroxyethylcellulose, hydroxethylmethylcellulose, carboxymethylcellulose, polyvinylacetate, polyalkeneoxide, polyalkeneglycol, polyoxyethylene-polyoxypropylene polymer (for example, Poloxamer™), zein, shellac, diethylaminoacetate (for example, AEA™), aminoalkylmethacrylate copolymer (for example, Eudragit E™), Sodium alginate, chitosan derivatives, gelatin, gum, poly-L-lysine and their mixtures.

Hydroxypropylcellulose, hydroxypropylmethylcellulose, polyvinylpyrrolidone, vinylpyrrolidone-vinylacetate copolymer, polyvinylalcohol, polyvinylalcohol-polyethyleneglycol copolymer and their mixtures are preferable if considering the solubility of the hydrophilic polymer and solid dispersion-forming ability with sibutramine free base. Polyvinylpyrrolidone is more preferable because of its high solubility if considering mass-production, and hydroxypropylcellulose and hydroxypropylmethylcellulose are more preferable if considering storage stability.

More preferably, the present invention also provides a sibutramine free base-containing solid dispersion wherein the solid dispersion is coated by non-hygroscopic or little hygroscopic hydrophilic polymer. In case that a hydrophilic polymer used for making a solid dispersion is hygroscopic, it has negative influence on stability of sibutramine free base, and this problem can be solved by coating the solid dispersion with non-hygroscopic or little hygroscopic polymer. Non-hygroscopic or little hygroscopic hydrophilic polymer includes, but is not limited to, hydroxypropylmethylcellulose, vinylpyrrolidone-vinylacetate copolymer, polyvinylalcohol, polyvinylalcohol-polyethyleneglycol copolymer and their mixtures. For example, a sibutramine free base-containing solid dispersion made with hygroscopic polyvinylpyrrolidone can be coated with hydroxypropylmethylcellulose to improve stability.

The present invention is also based on the surprising result that the improving effect of dissolution rate can be kept with only small amount of acid compared to the content of sibutramine free base, even if the improving effect increases according to the content of acid. This can be very advantageous when considering that it is preferable that the contents of additional ingredients except sibutramine free base is minimal in view of safety.

It is preferable that the content of the acid in the solid dispersion of the present invention is 0.1-20 moles per mole the sibutramine free base or 0.06-10 parts by weight per part by weight of the sibutramine free base.

In case that the content of the acid is less than 0.1 mole or 0.06 parts by weight, the dissolution rate of the solid dispersion according to pH may be worse than conventional preparations containing sibutramine hydrochloride monohydrate. In case that the content of the acid is more than 20 mole or 10 parts by weight, the stability of the composition comprising the solid dispersion may be worse because of the hygroscopic property of some acids.

It is preferable that the content of the hydrophilic polymer in the solid dispersion of the present invention is 0.125-30 parts by weight per part by weight of the sibutramine free base. In case that the content of the hydrophilic polymer is less than 0.125 parts by weight, making granules is so difficult that solid dispersions may have bad flowability, which makes following manufacturing processes difficult. In addition, in case that the content of the hydrophilic polymer is too low, the stability of the solid dispersion may be too bad to keep the improved dissolution rate during storage. In case that the content of the hydrophilic polymer is more than 30 parts by weight, the size of the preparation comprising the solid dispersion is too big to be taken, and it is difficult to make micro-environmental condition around sibutramine free base acidic because the content of the acid becomes small relatively.

The present invention also provides a pharmaceutical composition or preparation comprising the solid dispersion. The pharmaceutical composition or preparation can further comprise pharmaceutically acceptable excipients such as disintegrator, dilutor, flavor, colorant, lubricant, filler, etc., which are conventionally used to make an oral preparation (for example, tablet, granule, capsule and pellet).

The present invention also provides a method for manufacturing sibutramine free base-containing solid dispersion comprising (S1) making a solution wherein sibutramine free base, acid and hydrophilic polymer are dissolved; and (S2) drying the solution of (S1) step. The method of the present invention can easily prepare a solid dispersion wherein the acid and the sibutramine free base are minutely and uniformly mixed between uniformly entangled polymers.

More preferable, the present invention provides said method for manufacturing sibutramine free base-containing solid dispersion wherein the method further comprises (S3) coating the solid dispersion of (S2) step with non-hygroscopic or little hygroscopic polymer to more improve the stability of the solid dispersion. The non-hygroscopic or little hygroscopic polymer includes, but is not limited to, hydroxypropylmethylcellulose, vinylpyrrolidone-vinylacetate copolymer, polyvinylalcohol, polyvinylalcohol-polyethyleneglycol copolymer and their mixtures.

The solvent to make the solution of (S1) step includes, but is not limited to, water, ethanol, methanol, isopropylalcohol, dichloromethane, chloroform, acetone and their mixtures.

Drying of (S2) step can be performed with heated air, and methods to do such a drying are well known to those skilled in the art. It is preferable to do mixing such as stirring, shaking, rotating and so on during drying procedure to keep uniformity of the solution of (S1) step. Spray-dying method is more preferable in view of mass-production. Fluidized bed granulator, spray-dryer, fluidized bed dryer, C/F granulator, etc. can be used for spray-drying.

### MODE FOR THE INVENTION

Hereinafter, a preferred embodiment of the present invention will be described in detail. Prior to the description, it should be understood that various modifications are possible to the embodiments of the present invention, and it should be understood that the scope of the invention is not limited to the following embodiments. The embodiments are purposed to merely give better explanation of the invention to those ordinarily skilled in the art.

### Preparations of Examples 1˜6 and Comparative Examples 1˜2

Sibutramine free base-containing solid dispersions were prepared with various hydrophilic polymers. Sibutramine free base-containing solid dispersions and simple mixtures were prepared according to ingredients and contents of the below table 1.

For example, example 1 was prepared as follows: Sibutramine free base and citric acid were dissolved in 20□ of ethanol, and then hydroxypropylcellulose was added slowly to the solution with continuous stirring. After hydroxypropylcellulose was dissolved completely, lactose and silicon dioxide were added to the solution and the solution was mixed. Then, the final solution was dried at 40° C. with continuous stirring. After drying, the dried product was ground slightly and sieved with 30 mesh size of sieve to form the solid dispersion of example 1. Examples 2˜6 were prepared according to the same method as used in example 1.

Comparative examples 1-2 were prepared by simply mixing ingredients of the below table 1 and sieving the mixture with 30 mesh size of sieve.

TABLE 1

| | | | | | | | Comparative | |
|---|---|---|---|---|---|---|---|---|
| | Example | | | | | | example | |
| (Unit: gram) | 1 | 2 | 3 | 4 | 5 | 6 | 1 | 2 |
| Sibutramine free base | 1 | 1 | 1 | 1 | 1 | 1 | 1 | 1 |
| Citric acid | 0.76 | 0.76 | 0.76 | — | — | — | 0.76 | 0.76 |
| Fumaric acid | — | — | — | 0.45 | 0.45 | 0.45 | — | — |
| Hydroxypropylcellulose | 2 | — | — | 2 | — | — | 2 | — |
| Hydroxypropylmethylcellulose | — | 1 | — | — | 1 | — | — | 1 |
| Polyvinylpyrrolidone | — | — | 5 | — | — | 5 | — | — |
| Lactose | 18.7 | 18.7 | 18.7 | 18.7 | 18.7 | 18.7 | 18.7 | 18.7 |
| Silicon dioxide | 1.5 | 1.5 | 1.5 | 1.5 | 1.5 | 1.5 | 1.5 | 1.5 |

Hereinafter, unless otherwise stated, HPMC2910, Plasdone K29™ and Aerosil 200™ were used as hydroxypropylmethylcellulose, polyvinylpyrrolidone and silicone dioxide, respectively.

### Dissolution Tests of Examples 1˜6 and so on

Dissolution tests were performed with examples 1˜6, comparative examples 1˜2, sibutramine free base itself and Reductil™ (Abbott Korea Limited) containing 10□ of sibutramine hydrochloride monohydrate. To keep dissolution test condition similar, the contents of sibutramine in examples and comparative examples were evaluated with the below HPLC analysis condition and then the predetermined amount of examples, comparative examples and sibutramine free base itself were filled in one capsule to make capsules having 8.37□ of sibutramine free base. These capsules were used in dissolution tests.

500□ of pH 4.5 buffered solution [2.99 g of Sodium acetate trihydrate and 1.66□ of acetic acid anhydrous were dissolved in 1 L of distilled water and then pH of the solution was adjusted to pH 4.5±0.05] and 500□ of pH 6.8 buffered solution [118□ of 0.2 mol/L NaOH solution was added to 250□ of 0.2 mol/L K₂HPO₂, and then distilled water was added to make the solution 1000□] were used as dissolution media. Paddle method of dissolution test of the Korean Pharmacopoeia was used and paddle was rotated at 50 rpm. Dissolution test was performed for 45 minutes and sinker was also used.

For HPLC analysis, mixture (350:640:10) of acetonitrile, water and tetrahydrofuran was used as mobile phase and ion pair reagent PIC B5 (Low UV) was added to the mobile phase to adjust retention time of peak, and then pH was adjusted to 3.0 with phosphoric acid. Wavelength for determination was 229□ and flow rate was 1.5□/min and injection volume was 20□.

Results in pH 4.5 and pH 6.8 buffered solutions were shown in table 2 and 3, respectively. Results were shown as percent (%) of sibutramine dissolved into dissolution media from test samples compared to the total amount of sibutramine contained in each sample.

TABLE 2

| <pH 4.5 solution> (unit: %) | 15 min | 30 min | 45 min |
|---|---|---|---|
| Sibutramine free base | 62 | 80 | 88 |
| Comparative example 1 | 77 | 86 | 89 |
| Comparative example 2 | 82 | 95 | 102 |
| Reductil | 79 | 96 | 99 |
| Example 1 | 92 | 98 | 100 |
| Example 2 | 95 | 98 | 100 |
| Example 3 | 92 | 96 | 97 |
| Example 4 | 96 | 102 | 103 |
| Example 5 | 89 | 99 | 101 |
| Example 6 | 102 | 102 | 101 |

As shown in table 2, all test samples including sibutramine free base itself and comparative examples showed high dissolution rates in pH 4.5 buffered solution.

TABLE 3

| <pH 6.8 solution> (unit: %) | 15 min | 30 min | 45 min |
|---|---|---|---|
| Sibutramine free base | 0 | 0 | 0 |
| Comparative example 1 | 12 | 15 | 19 |
| Comparative example 2 | 11 | 17 | 21 |
| Reductil | 35 | 42 | 46 |
| Example 1 | 65 | 70 | 71 |
| Example 2 | 70 | 72 | 73 |
| Example 3 | 62 | 70 | 69 |
| Example 4 | 62 | 68 | 69 |
| Example 5 | 61 | 65 | 67 |
| Example 6 | 60 | 63 | 64 |

As shown in table 3, in dissolution tests using pH 6.8 buffered solution, sibutramine free base did not practically dissolve and comparative examples made by simple mixing of sibutramine free base, acid and hydrophilic polymer showed 20% of low dissolution rate at 45 minutes, but examples 1 and 2 having the same ingredients and contents as comparative examples showed more than 70% of dissolution rate at 45 minutes, and these dissolution rates of examples 1 and 2 were much higher than that of Reductil™ containing sibutramine hydrochloride monohydrate. In addition, examples 3-6 using other polymer and/or other acid showed very high dissolution rates in pH 6.8 buffered solution.

### Preparations of Examples 7˜15

Sibutramine free base-containing solid dispersions were prepared with various acids as shown in the below table 4 according to the same method as used in example 1.

TABLE 4

| | Example | | | | | | | | |
|---|---|---|---|---|---|---|---|---|---|
| (Unit: gram) | 7 | 8 | 9 | 10 | 11 | 12 | 13 | 14 | 15 |
| Sibutramine free base | 1 | 1 | 1 | 1 | 1 | 1 | 1 | 1 | 1 |
| Lactic acid | 0.33 | — | — | — | — | — | — | — | — |
| Ttartaric acid | — | 0.59 | — | — | — | — | — | — | — |
| Succinic acid | — | — | 0.47 | — | — | — | — | — | — |
| Maleic acid | — | — | — | 0.45 | — | — | — | — | — |
| Malic acid | — | — | — | — | 0.53 | — | — | — | — |
| L-glutamic acid | — | — | — | — | — | 0.58 | — | — | — |
| Ppalmitic acid | — | — | — | — | — | — | 1.01 | — | — |
| Oxalic acid | — | — | — | — | — | — | — | 0.5 | — |
| L-aspartic acid | — | — | — | — | — | — | — | — | 0.52 |
| Hydroxypropylmethylcellulose | 1 | 1 | 1 | 1 | 1 | 1 | 1 | 1 | 1 |
| Lactose | 22 | 22 | 22 | 22 | 22 | 22 | 22 | 22 | 22 |
| Silicon dioxide | 1.5 | 1.5 | 1.5 | 1.5 | 1.5 | 1.5 | 1.5 | 1.5 | 1.5 |

### Dissolution Tests of Examples 7˜15 and so on

Dissolution tests on examples 7-15, sibutramine free base itself and Reductil™ were performed according to the same method as used in dissolution test on example 1. Distilled water and pH 6.8 buffered solution were used as dissolution media Results of pH 6.8 buffered solution and distilled water were shown in table 5 and 6, respectively.

TABLE 5

| <pH 6.8 solution> (unit: %) | 15 min | 30 min | 45 min |
|---|---|---|---|
| Sibutramine free base | 0 | 0 | 0 |
| Reductil | 35 | 42 | 46 |
| Example 7 | 35 | 44 | 50 |
| Example 8 | 38 | 46 | 58 |
| Example 9 | 47 | 54 | 58 |
| Example 10 | 32 | 41 | 45 |
| Example 11 | 41 | 47 | 52 |
| Example 12 | 21 | 27 | 31 |
| Example 13 | 32 | 43 | 46 |
| Example 14 | 84 | 68 | 73 |
| Example 15 | 22 | 25 | 27 |

TABLE 6

| <Distilled water> (unit: %) | 15 min | 30 min | 45 min |
|---|---|---|---|
| Sibutramine free base | 0 | 0 | 0 |
| Reductil | 76 | 80 | 82 |
| Example 7 | 54 | 68 | 70 |
| Example 8 | 95 | 101 | 103 |
| Example 9 | 74 | 78 | 79 |
| Example 10 | 75 | 82 | 84 |
| Example 11 | 73 | 76 | 81 |
| Example 12 | 39 | 47 | 51 |
| Example 13 | 10 | 16 | 19 |
| Example 14 | 95 | 97 | 99 |
| Example 15 | 39 | 40 | 44 |

In comparison with sibutramine free base, the improved dissolution rate had nothing to do with the kind of acid. However, example 13 using palmitic acid showed lower dissolution rate than Reductil™ in dissolution test using distilled water, and example 12 using glutamic acid and example 15 using aspartic acid showed lower dissolution rate that Reductil™ in both distilled water and pH 6.8 buffered solution. It means that there are more preferable acids for improving dissolution rate.

### Preparations of Examples 16˜19

Solid dispersions having various content ratios of acid were prepared by increasing the content of acid per mole or part by weight of sibutramine free base to determine the amount of acid needed, as shown in the below table 7 according to the same method as used in example 1.

TABLE 7

| | | | Example | Example | |
|---|---|---|---|---|---|
| (Unit: gram) | Example 3 | Example 16 | 17 | 18 | Example 19 |
| Sibutramine free | 1 (3.57) | 1 (3.57) | 1 (3.57) | 1 (3.57) | 1 (3.57) |
| base (mole × 10³) | | | | | |
| Citric acid (mole × 10³, mole | 0.760 (3.96, 1.1) | 0.340 (1.79, 0.5) | 0.204 (1.06, 0.3) | 0.136 (0.72, 0.2) | 0.068 (0.36, 0.1) |
| ratio*) | | | | | |
| Polyvinylpyrrolidone | 5 | 5 | 5 | 5 | 5 |
| Lactose | 18.7 | 18.7 | 18.7 | 18.7 | 18.7 |
| Silicon dioxide | 1.5 | 1.5 | 1.5 | 1.5 | 1.5 |

In table 7, “mole ratio*” means the ratio of mole of the citric acid contained in solid dispersion per mole of sibutramine free base in solid dispersion.

### Dissolution Tests of Examples 16˜19 and so on

Dissolution test on examples 3 and 16˜19, sibutramine free base itself and Reductil™ were performed according to the same method as used in dissolution test on example 1. Results were shown in table 8.

TABLE 8

| <pH 6.8 solution> (unit: %) | 15 min | 30 min | 45 min |
|---|---|---|---|
| Sibutramine free base | 0 | 0 | 0 |
| Reductil | 35 | 42 | 46 |
| Example 3 | 62 | 70 | 69 |
| Example 16 | 59 | 63 | 65 |
| Example 17 | 53 | 60 | 62 |
| Example 18 | 51 | 57 | 59 |
| Example 19 | 48 | 55 | 58 |

As shown in table 8, the decrease of the content of citric acid lowered slightly the improving effect of dissolution rate. However, the solid dispersion of the present invention showed much higher dissolution rate than the conventional preparation containing sibutramine hydrochloride monohydrate even if the content of citric acid is very small (for example, 0.1 mole or 0.068 parts by weight per mole or part by weight of sibutramine free base in example 19).

This result that dissolution rate does not in proportion to the content of acid is very surprising, which means that the solid dispersion of the present invention can make micro-environmental condition around sibutramine free base acidic even in case that the content of acid is very small.

### Preparation of Example 20

Example 20 was prepared to evaluate mass-production ability of the solid dispersion of the present invention and the dissolution rate of mass-produced solid dispersion. 30.5 g of sibutramine free base and 23.2 g of citric acid were dissolved in 620□ of ethanol, and then 91.4 g of polyvinylpyrrolidone was added slowly to the solution and dissolved. 609.3 g of lactose and 20.1 g of silicon dioxide (aerosol 200™, Degussa, Germany) were fluidized in the bed of fluidized bed granulator (GX-20, Freund, Japan), and then the solution prepared above was sprayed in the following conditions to form example 20.
Operating condition of the fluidized bed granulator

Temperature and amount of inlet air: 65° C., 0.5 L/min

Temperature and amount of slit air: 65° C., 0.4 L/min

Speed of revolution of rotor: 250 rpm

### Preparation of Example 21

Solid dispersion containing sibutramine free base, citric acid, polyvinylpyrrolidone, lactose and silicon dioxide was prepared according to the same method as used in example 20. Then, 59.1 g of hydroxypropylmethylcellulose, 9 g of polyethyleneglycol 6000 and 15 g of talc were dissolved or suspended in mixture of 700□ of ethanol and 300□ of distilled water to make a coating solution. After fluidizing the solid dispersion granules in the fluidized bed granulator, the coating solution was sprayed in the following conditions to make a coated solid dispersion.
Operating condition of the fluidized bed granulator

Temperature and amount of inlet air: 65° C., 0.3 L/min

Temperature and amount of slit air: 65° C., 0.3 L/min

Speed of revolution of rotor: 250 rpm

### Dissolution tests of examples 20 and 21

Dissolution tests on solid dispersions made in examples 20 and 21 were performed according to the same method as used in dissolution test on example 1. Results were shown in table 9.

TABLE 9

| | Example 20 | Example 20 | Example 21 |
|---|---|---|---|
| (Unit: %) | pH 4.5 solution | pH 6.8 solution | pH 6.8 solution |
| 15 min | 97 | 60 | 61 |
| 30 min | 97 | 62 | 60 |
| 45 min | 98 | 62 | 63 |

As shown in table 9, mass-produced solid dispersions of the present invention showed high dissolution rates.

### Evaluation of Mass-Production Ability According to the Content of Polymer

Sibutramine free base-containing solid dispersions were prepared according to the same method as used in example 20 except that 61, 30.5, 15.3 or 7.7 g of polyvinylpyrrolidone was used instead of 91.4 g of polyvinylpyrrolidone.

In case that the hydrophilic polymer was comprised in a small amount per the amount of sibutramine free base, it was difficult to make granules even if the dissolution rate of the solid dispersion comprising a small amount of polymer was higher than the conventional preparation containing sibutramine hydrochloride monohydrate in dissolution test using pH 6.8 buffered solution. In case using 7.7 g of polyvinylpyrrolidone, formed granules were easily broken. This caused lowering of flow-ability of granules, which made it difficult to fill capsules.

### Stability Test on Content and Dissolution of Example 20

Stability test was performed at 40° C., 75% RH for 2 months with sibutramine free base-containing solid dispersion made in example 20. After 2 months, the content and dissolution rate were determined according to the same method as used in evaluation of example 1. Changes of content and dissolution rate were shown in table 10 and 11, respectively.

TABLE 10

| | Before starting | |
|---|---|---|
| Content | stability test | After 2 months |
| Example 20 (40° C., 75% RH) | 100% | 99.7% |

TABLE 11

| Dissolution rate | | Before starting | |
|---|---|---|---|
| (pH 6.8 solution) | | stability test | After 2 months |
| Example 20 (40° C., | 15 min | 60 | 61 |
| 75% RH) | 30 min | 62 | 64 |
| | 45 min | 62 | 65 |

As shown in table 10 and 11, the content and dissolution rate of sibutramine free base-containing solid dispersion were not changed after 2 months of storage in accelerated condition. These results mean that the solid dispersion of the present invention is very stable.

### Stability Tests on Appearance and Content of Examples 20˜21 and so on

Appearance and content stability test were performed at 60° C., 75% RH with solid dispersions of examples 20 and 21. Comparative example 3 was prepared by simply mixing the ingredients of example 20 having the same contents without making the solid dispersion. Results were shown in table 12 and evaluation criteria of appearance are as follows:

—Evaluation criteria of appearance—
++++: no change of appearance and good flow-ability
+++: small amount of aggregates of granules and normal flow-ability
++: aggregates of granules and bad flow-ability
+: hardened aggregates and no flow

TABLE 12

| | Appearance/content(%) | | | | |
|---|---|---|---|---|---|
| | 0 wk | 1 wk | 2 wks | 3 wks | 4 wks |
| Example 20 | ++++100.3% | ++++99.9% | +++99.4% | +++98.3% | +++98.1% |
| Comparative example 3 | ++++100.1% | +++93.0% | ++90.5% | +88.2% | +86.8% |
| Example 21 | ++++100.1% | ++++99.9% | ++++100.0% | ++++99.1% | ++++99.2% |

As shown in table 12, the solid dispersion of example 20 according to the present invention showed better stability in both appearance and content than comparative example 3 having the same ingredients and contents as example 20. Example 20 showed a little aggregation of granules over time, which is thought to be caused by the hygroscopic property of polyvinylpyrrolidone. This aggregation could be blocked by coating the granules with hydroxypropylmethylcellulose without change of dissolution rate.

### Preparations of Examples 22˜23

Sibutramine free base-containing solid dispersions were prepared with inorganic acid instead of organic acid according to the same method as used in example 1, as shown in the table 13.

TABLE 13

| (Unit: gram) | Example 22 | Example 23 |
|---|---|---|
| Sibutramine free base (mole × 10³) | 1 (3.57) | 1 (3.57) |
| Phosphoric acid (mole × 10³, | 0.388 (3.96, 1.1) | 0.175 (1.79, 0.5) |
| mole ratio*) | | |
| Polyvinylpyrrolidone | 5 | 5 |
| Lactose | 18.7 | 18.7 |
| Silicone dioxide | 1.5 | 1.5 |

In table 13, “mole ratio*” means the ratio of mole of the phosphoric acid contained in solid dispersion per mole of sibutramine free base in solid dispersion.

### Dissolution Tests of Example 22 and 23

Dissolution tests on example 22 and 23 were performed in distilled water, pH 4.0 buffered solution and pH 6.8 buffered solution according to the same method as used in the dissolution test on example 1. Results were shown in table 14, 15 and 16, respectively.

TABLE 14

| <pH 4.5 solution> (Unit: %) | 15 min | 30 min | 45 min |
|---|---|---|---|
| Example 22 | 101 | 102 | 101 |
| Example 23 | 101 | 101 | 101 |

TABLE 15

| <pH 6.8 solution> (Unit: %) | 15 min | 30 min | 45 min |
|---|---|---|---|
| Example 22 | 61 | 66 | 67 |
| Example 23 | 57 | 61 | 64 |

TABLE 16

| <Distilled water> (Unit: %) | 15 min | 30 min | 45 min |
|---|---|---|---|
| Example 22 | 94 | 95 | 95 |
| Example 23 | 76 | 79 | 80 |

As shown in table 14, 15 and 16, solid dispersions using phosphoric acid as acid showed much higher dissolution rates than both sibutramine free base itself and the conventional preparations containing sibutramine hydrochloride monohydrate. In addition, solid dispersion containing 0.5 mole or 0.175 parts by weight of phosphoric acid compared to the amount of sibutramine free base also showed higher dissolution rates than Reductil™ similar to dissolution results of examples 16˜19.

### INDUSTRIAL APPLICABILITY

The present invention provides a solid dispersion comprising sibutramine free base, acid and hydrophilic polymer; a pharmaceutical composition comprising the solid dispersion; and a manufacturing method thereof. Sibutramine free base-containing solid dispersion according to the present invention shows higher dissolution rate in high pH medium than conventional preparations containing sibutramine free base or sibutramine hydrochloride monohydrate. The solid dispersion according to the present invention is very stable compared to conventional preparations containing sibutramine free base.

1. A sibutramine free base-containing solid dispersion wherein sibutramine free base, acid and hydrophilic polymer are uniformly dispersed.

2. The solid dispersion of claim 1, wherein the acid is at least one selected from the group consisting of citric acid, fumaric acid, lactic acid, tartaric acid, succinic acid, maleic acid, malic acid, oxalic acid and phosphoric acid.

3. The solid dispersion of claim 1, wherein the hydrophilic polymer is at least one selected from the group consisting of hydroxypropylcellulose, hydroxypropylmethylcellulose, polyvinylpyrrolidone, vinylpyrrolidone-vinylacetate copolymer, polyvinylalcohol and polyvinylalcohol-polyethyleneglycol copolymer.

4. The solid dispersion of claim 1, wherein the acid is comprised in an amount of 0.1-20 moles per mole of the sibutramine free base.

5. The solid dispersion of claim 1, wherein the acid is comprised in an amount of 0.06-10 parts by weight per part by weight of the sibutramine free base.

6. The solid dispersion of claim 1, wherein the hydrophilic polymer is comprised in an amount of 0.125-30 parts by weight per part by weight of the sibutramine free base.

7. The solid dispersion of claim 1, wherein the solid dispersion is coated by at least one selected from the group consisting of hydroxypropylmethylcellulose, vinylpyrrolidone-vinylacetate copolymer, polyvinylalcohol and polyvinylalcohol-polyethyleneglycol copolymer.

8. A pharmaceutical preparation comprising the solid dispersion of any of claims 1-7.

9. A method for manufacturing sibutramine free base-containing solid dispersion comprising (S1) making a solution wherein sibutramine free base, acid and hydrophilic polymer are dissolved; and (S2) drying the solution of (S1) step.

10. The method of claim 9, wherein the acid is at least one selected from the group consisting of citric acid, fumaric acid, lactic acid, tartaric acid, succinic acid, maleic acid, malic acid, oxalic acid and phosphoric acid.

11. The method of claim 9, wherein the hydrophilic polymer is at least one selected from the group consisting of hydroxypropylcellulose, hydroxypropylmethylcellulose, polyvinylpyrrolidone, vinylpyrrolidone-vinylacetate copolymer, polyvinylalcohol and polyvinylalcohol-polyethyleneglycol copolymer.

12. The method of claim 9, wherein the acid is comprised in an amount of 0.1-20 moles per mole of the sibutramine free base.

13. The method of claim 9, wherein the acid is comprised in an amount of 0.06-10 parts by weight per part by weight of the sibutramine free base.

14. The method of claim 9, wherein the hydrophilic polymer is comprised in an amount of 0.125-30 parts by weight per part by weight of the sibutramine free base.

15. The method of claim 9, wherein the method further comprises (S3) coating the solid dispersion of (S2) step with at least one selected from the group consisting of hydroxypropylmethylcellulose, vinylpyrrolidone-vinylacetate copolymer, polyvinylalcohol and polyvinylalcohol-polyethyleneglycol copolymer.
